(19) United States
(12) Patent Application Publication
(10) Pub. No.: US 2020/0375746 A1
(43) Pub. Date: Dec. 3, 2020

(54) Methods, Compositions and Articles for Improving Joint Lubrication

(71) Applicant: Maguire Abbey, LLC, Wilmington, DE (US)

(72) Inventors: Joseph P. Laico, New City, NY (US); Michael P. Greuel, Solon, OH (US)

(21) Appl. No.: 16/808,931

(22) Filed: Mar. 4, 2020

Related U.S. Application Data
(63) Continuation of application No. PCT/US2018/049590, filed on Sep. 5, 2018.
(60) Provisional application No. 62/554,525, filed on Sep. 5, 2017.

(51) Int. Cl. A61F 2/30; A61L 27/58; A61L 27/54; A61K 9/16

(52) U.S. Cl. CPC A61F 2/30; A61L 27/58; A61F 2002/30673; A61K 9/1647; A61L 2300/236; A61L 27/54

(57) ABSTRACT

Articles for increasing lubrication of a joint are described herein. The articles include resorbable, biocompatible particles which may include at least one polymer and are capable of increasing fluid movement within the joint compared to synovial fluid, viscosupplemental fluid, or combinations thereof. In some embodiments, the at least one polymer has a glass transition temperature within a joint of less than about 37° C. A composition for increasing lubrication of a joint is also disclosed. The composition includes the resorbable, biocompatible particles and a carrier fluid. Methods of lubricating a joint and treating disease affecting the joint such as osteoarthritis are also described herein. The methods include introducing the resorbable, biocompatible particles into a joint.

### CROSS-REFERENCE TO RELATED APPLICATIONS

This Application is a continuation application claiming priority under 35 U.S.C. §§ 120 and 365(c) to International PCT application No. PCT/US2018/049590 which designates the United States and was filed in the English language, which International PCT Application No. PCT/US2018/049590 claims the benefit under 35 U.S.C. § 119(e) to U.S. provisional patent application No. 62/554,525, and this incorporates by reference herein the entire disclosures of these applications in their entirety.

### BACKGROUND OF THE INVENTION

### Field of the Invention

The present invention is directed to assisting in lubricating the joints of mammals and methods of treating osteoarthritis and joint-related pain and ailments.

### Description of Related Art

Synovial joints such as hip, knee, shoulder and ankle joints are surrounded by an envelope or synovial capsule. The inner layer of the synovial capsule is called a synovial membrane which produces synovial fluid. The fluid is partially stored within the joint cartilage and the remaining fluid circulates freely within the synovial capsule. The capsule maintains the fluid within the joint. In a hip joint, a ring of soft tissue called the acetabular labrum aids in maintaining the fluid in the femoral-acetabular interface. The fluid lubricates and thus reduces friction inside of the joint. In ball and socket synovial joints, the fluid lubricates the ball and socket interface, particularly during movement. For example, the wringing action of the synovial capsule in a hip joint, particularly during flexion and extension movement of the joint, and the paddling action of the femoral neck combine to pump synovial fluid into and across the femoral-acetabular interface thus lubricating the joint. The synovial fluid also cushions the joints during movement, provides oxygen and nutrients to the joint cartilage and removes carbon dioxide and metabolic waste.

Synovial fluid is generally composed of hyaluronic acid, lubricin, proteinases, and collagenases. The hyaluronic acid imparts anti-inflammatory and pain-reducing properties to the normal synovial fluid and contributes to joint lubrication and cushioning during movement. Synovial fluid also exhibits non-Newtonian flow characteristics and thixotropy where the fluid viscosity decreases over time under stress due to movement.

A lack of synovial fluid within the joint, particularly within the ball and socket interface, can aggravate arthritic conditions. Osteoarthritis, the wear and tear of aging, and other injuries or ailments can cause irregularity of the joint surface. In a hip joint, osteoarthritis can also cause fraying of the acetabular labrum resulting in the loss of its gasket-like sealing property. The fraying of the labrum allows migration of the synovial fluid away from the femoral-acetabular interface. Gravity also acts on vertical synovial joints such as hip joints by drawing the synovial fluid downward and away from the femoral-acetabular interface. Moreover, the stress and/or inflammation in synovial joints over time reduce the viscosity of the fluid, making it a less effective lubricant and more difficult for the fluid to effectively coat the joint interface. This reduction in synovial fluid flow in the joint interface often results in further reduction in the sealing capacity of the labrum and roughening or incongruity of the joint interface causing increased pain and stiffness in the joint. The pain and stiffness causes a decrease in the motion of the joint resulting in a loss of the pumping action and decrease in the flow of the synovial fluid in the joint interface. This can eventually lead to joint replacement surgery.

To address this problem, artificial lubricants have been developed to replace and/or supplement the lubricating and cushioning action of the synovial fluid in the joint. These lubricants are generally referred to as viscosupplements and generally include hyaluronic acid. However, the degradation of the acetabular labrum associated with osteoarthritis can result in leakage and decreased flow of the viscosupplements. Thus, multiple viscosupplement treatments can be required.

Others have proposed the injection of biodegradable microparticles containing therapeutic agents into the arthritic joints. U.S. Patent Application Publication Nos. 2007/0141160 and 2010/0016257 to Brown, et al. disclose a method of treatment that includes intra-articular injection of biodegradable, polymer microparticles in a carrier vehicle. The microparticles are 5 to 150 microns and may be introduced with a carrier vehicle such as one including a therapeutic agent, for example, hyaluronic acid. The composition is injected into the intra-articular space of a joint to treat joint pain associated with osteoarthritis.

Other treatments to address this issue include joint replacement surgery, arthroscopic surgery, medication and physical therapy. Joint replacement surgery includes replacement of the joint with a prosthetic implant. The prosthetic implant may be constructed of various materials including metal and polymer materials. In addition the typical health risks associated with major joint surgery in older patients, risks and complications of the procedure include infection, dislocation, loosening, or impingement of the implant. In hip replacement surgery, the risks also include fractures of the femur. Moreover, the implant may wear over time causing dissemination of metal and polymer debris within the joint and body, in general.

There exists a need in the art for other innovative methods to improve joint lubrication and thus address the degradation and reduction in the circulation of synovial fluid associated with aging, osteoarthritis, injuries and other ailments. Such methods will preferably relieve pain and extend joint life to avoid the drawbacks associated with joint replacement surgery and to improve on existing treatments.

### BRIEF SUMMARY OF THE INVENTION

Applicant herein has addressed issues in the prior art in applicant's U.S. Pat. No. 9,186,377. In that invention, biocompatible, resorbable polymers and copolymers are used to form particles sufficient to operate to increase fluid movement within a joint. The particles preferably have a Young's Modulus and Poisson's ratio as well as an average density that allow them to function along with synovial fluid or other lubricant additives to push and move fluid through the joint space.

Polymers identified in embodiments in U.S. Pat. No. 9,186,377 include poly(alpha-hydroxy acid) polymers such as poly(glycolic acid) (PGA), copolymers of lactic acid and glycolic acid (PLGA), polyoxalates, polycaprolactone (PCL), copolymers of caprolactone and lactic acid (PCLA), poly(ether ester) multiblock copolymers based on polyethylene glycol and poly(butylene terephthalate), tyrosine-derived polycarbonates, poly(hydroxybutyrate), poly(alkylcarbonate), poly(orthoesters), polyesters, poly(hydroxyvaleric acid), poly(malic acid), poly(tartaric acid), poly(acrylamides), polyanhydrides, and polyphosphazenes. The copolymers may be random, alternating, block, or graft copolymers. Suitable polymeric materials also include waxes such as glycerol mono- and distearate and the blends thereof. Such polymers may also be combined into blends, alloys or copolymerized with one another and also functionalized, with a particular focus on copolymers of L-lactide and caprolactone such as poly(L-lactide-co-caprolactone) with an L-lactide to caprolactone monomer ratio of 70:30 or less.

In further working with the materials noted above, the applicant determined that additional, biocompatible resorbable elastomer materials which may or may not be modified for lubrication enhance the beneficial effects of the invention of U.S. Pat. No. 9,186,377.

The present invention thus includes articles for and a method for increasing lubrication of a joint. The articles are resorbable, biocompatible particles, which preferably have a glass transition temperature (T_{g}) within the joint of less than about 37° C. The particles are capable of increasing fluid movement within the joint compared to synovial fluid, viscosupplemental fluid, or combinations thereof. The particles preferably have an average particle size of about 0.5 millimeters to about 5 millimeters. The average particle size is most preferably about 3 millimeters.

In preferred embodiments, herein, the particles have a Young's Modulus of about 1 megapascal to about 500 megapascals, or about 10 megapascals to about 500 megapascals, and also have a Poisson's ratio of about 0.1 to about 0.5, as well as an average density greater than the average density of the fluid within the joint. In more preferred embodiments, the particles have a Young's Modulus of about 1 megapascal to 100 megapascals, or about 10 megapascals to about 100 megapascals. In the most preferred embodiment, the particles have a Young's Modulus of about 1 megapascal to about 30 megapascals, and in another preferred embodiment, about 10 to about 30 megapascals. A most preferred embodiment having a Young's Modulus of about 1 to about 30 megapascals also has Poisson's ratio of about 0.3, and an average density of about 1.2 g/ml.

In yet another preferred embodiment, the particles resorb in vivo in about 3 to about 18 months. The particles more preferably resorb in vivo in about 12 to about 18 months, although results may be varied for different end effects and based upon the polymer selected.

One preferred embodiment herein includes particles formed of polymers and copolymers of lactic acid and caprolactone. When using such polymers, the particles are more preferably formed of poly(L-lactide-co-caprolactone) wherein the monomer ratio of L-lactide to caprolactone ranges from about 70:30 to about 5:95. The inherent viscosity of the particles is also preferably about 0.15 to about 3.0 deciliters per gram. Moreover, the particles are preferably spherical.

The particles can also preferably be formed from resorbable, biocompatible elastomers formed from copolymerization of polyols, including, without limitation, as glycerol, erythritol, mannitol, and the like, with dicarboxylic acids, such as but not limited to oxalic acid, malonic acid, succinic acid, glutaric acid, adipic acid, pimelic acide, suberic acid, azelaic acid, sebacic acid, and the like. Such copolymers which may be elastomers and/or have elastomeric properties may be further copolymerized with lactic acids and their copolymers as well. Examples of preferred dicarboxylic acid-polyol copolymer bioresorbable materials include poly(glycerol sebacate) (PGS), which imparts improved recovery to the particles upon deformation and enhances the retention of physical properties over the course of degradation, in-vivo, as well as copolymers thereof, as well as poly(glycerol sebacate lactic acid) (PGSL) and similar polymers and elastomers, with PGS being preferred.

Such bioresorbable preferred materials may be further modified to create preferred physical or chemical properties by cross-linking by polymerizing with functionalized monomers or other chain modification through grafting or otherwise providing functional groups to through functionalizing one or more of the monomers to form a functional polymer. Examples include a modified or functionalized poly(L-lactide-co-caprolactone, modified or functionalized PGS or PGSL among others. Such modified polymers may be used to form a bulk particle having such modification throughout the particle or the particles formed and then the surface modified by a surface functionalization.

In a preferred embodiment herein, particles formed from polymers such as poly(L-lactide-co-caprolactone), PGS or PGSL as noted above may also be modified during or after polymerization and/or during or after formation of the particles contemplated herein. In one embodiment, such polymers may also be modified by incorporating into the bulk and/or the surface thereof bio-lubricious compounds, such as proteoglycan 4 (also known as lubricin), other glycoproteins, hyaluronic acid, phospholipids and/or polymeric synthetic joint lubricants. The presence of bio-lubricious compounds on the surface of the particles in certain embodiments enhances frictional properties, resulting in improved movement within the joint and the mitigation of impingement. The presence of bio-lubricious compounds in the bulk of the particles, in the case of surface eroding materials such as PGS, can further be employed to provide replenishment of a resulting bio-lubricious PGS as the particle degrades.

Notably, the presence of biolubricious compound(s), such as lubricin and hyaluronic acid compounds, on the surface and/or in the bulk of the particles increases particle lubricity both between the individual particles themselves as well as between the particles and other intra-articular structures that may impede the motion of the particles. Such structures include, for example, but are not limited to, the capsule, cartilaginous and bony structures, such as the femoral neck (in the case of a hip joint), the tibial plateau and femoral condyles (in the case of the knee), the humeral neck (in the case of the shoulder), the talus and distal tibia and fibula (in the case of the ankle); soft tissue damaged by the arthritic process (such as the labrum in the hip and shoulder, and the menisci in the case of the knee); other irregularly shaped structures that may have arisen as a result of the arthritic disease process such as cartilage defects; and loose bodies. This enhanced lubricity would augment the purpose of the particles to drive the synovial/hyaluronic acid enhanced fluid across the joint interfaces.

As noted above, in preferred embodiments herein the particles comprise at least one polymer selected from poly(L-lactide-co-caprolactone, poly(glycerol sebacate), and poly(glycerol sebacate lactic acid), and preferably are particles that comprise poly(glycerol sebacate). The particles are also preferably elastomers or provide elastomeric properties in use in a joint.

In one embodiment, the at least one polymer in the particles preferably incorporates at least one bio-lubricious compound, wherein the bio-lubricious compound may be at least one of lubricin and hyaluronic acid. In one embodiment, the at least one bio-lubricious compound is incorporated into the bulk of the at least one polymer. The at least one bio-lubricious compound may also be, or alternatively may be, grafted and/or chemically attached to the at least one polymer on a surface of the particles.

A composition for increasing lubrication of a joint that includes resorbable, biocompatible particles as noted above and/or that has a T_{g} within a joint of less than about 37° C. The particles are capable of increasing fluid movement within the joint compared to use of synovial fluid, viscosupplemental fluid, or combinations thereof, and a carrier fluid is also disclosed herein. The carrier fluid preferably includes one or more of synovial fluid, viscosupplemental fluid and/or combinations thereof. The composition may also include at least one therapeutic agent such as hyaluronic acid, modified hyaluronic acid, anti-inflammatory medication such as steroids, non-steroidal anti-inflammatory agents, and numbing agents such as lidocaine.

The present invention further includes a method of lubricating a joint that includes introducing particles which may be formed of the materials as noted above into a joint. The particles are capable of increasing fluid movement within the joint compared to synovial fluid, viscosupplemental fluid, or combinations thereof and are formed of a resorbable, biocompatible material, which in one embodiment has a T_{g} within the joint of less than about 37° C. The particles are preferably introduced into the joint with a cannula. The inside diameter of the cannula is preferably about 2 millimeters to about 6 millimeters and more preferably about 4 millimeters to about 6 millimeters. The particles are also preferably introduced into the joint by arthroscopic visualization, x-ray-guided insertion, radiographically-guided insertion, sonographically-guided insertion or combinations thereof.

The method described above is preferably applied to synovial joints such as a hip, a knee, a shoulder, an ankle, an elbow, a wrist, a toe, a finger, and a spinal facet joint. The method may also be applied to a prosthetic implant or an arthritic joint or otherwise damaged joint.

In one embodiment, the invention includes a method of lubricating a joint comprising introducing particles into a joint, wherein the particles are capable of increasing fluid movement within the joint compared to synovial fluid, viscosupplemental fluid, or combinations thereof and are formed of a resorbable, biocompatible material, wherein the particles incorporate at least one biolubricious compound.

In that embodiment, the resorbable biocompatible material may comprise at least one reactive functional group and the at least one biolubricious compound is preferably incorporated into a surface of the particles by a grafting and/or surface modification reaction using a difunctional compound to crosslink at the least one functional group on the resorbable biocompatible material with a functional group on the at least one biolubricious compound at the surface of the particle. The resorbable biocompatible material may also be formed using at least one functionalized monomer capable of reacting with the at least one biolubricious compound so as to attach the at least one biolubricious compound to at least one location along a polymer chain of the resorbable biocompatible material before forming the particles. Further, the at least one biolubricious compound may be combined with the resorbable biocompatible material in a solvent-based reaction or latex polymerization reaction.

In a further embodiment, the at least one biolubricious compound may be combined with the resorbable biocompatible material prior to formation of the particles through at least one of mixing and/or blending. The at least one biolubricous compound may also be combined with the resorbable biocompatible material by swelling the particles with a solution comprising the biolubricious compound. The particles may be formed by at least one of a melt-processing process, a thermally cured condensation reaction process, a polymerization process initiated thermally, or initiated by irradiation with ultraviolet, e-beam, gamma or other radiation, a solvent-based process, cryoformation or latex polymerization.

The present invention further includes a method for treating a disease such as osteoarthritis that causes irregularity of the joint surfaces or breakdown of the soft tissue in the joint by introducing particles into a diseased joint, wherein the particles are capable of increasing fluid movement within the joint compared to synovial fluid, viscosupplemental fluid, or combinations thereof and are formed of a resorbable, biocompatible material such as those noted above, and which in one embodiment have a T_{g} within the joint of less than about 37° C.

### DETAILED DESCRIPTION OF THE INVENTION

The resorbable, biodegradable particles of the present invention increase the lubrication within a joint when introduced into the intra-articular space of the joint compared to synovial fluid, viscosupplemental fluid, or combinations thereof. The increase in fluid movement results in improved lubrication of the joint thus providing treatment of osteoarthritis and improved lubrication of prosthetic implants.

The particles of the present invention are preferably constructed from materials that preferably have a T_{g} within the joint of less than the normal body temperature of about 37° C. so that the particles are soft enough to prevent impingement within the joint interface. The fluid within the joint may have a plasticizing effect on the particles and thus reduce their T_{g} in-vivo. Accordingly, particles with a T_{g} outside of the body greater than 37° C. may still be suitable for the present invention.

The particles are sized so that they can effectively increase the fluid movement within the joint while limiting impingement in the joint interface. The average particle size of the present invention is preferably about 0.5 millimeters to about 5 millimeters. The average particle size is most preferably about 3 millimeters. The particles are preferably uniformly sized. However, significant particle size variations are also acceptable. The particle size may vary depending on the size of the device used to introduce the particles into the joint, the mass required to increase fluid motion within the joint, and volume of the joint space.

The physical parameters that affect the ability of the particles to increase fluid movement within a joint include, but are not limited to, Young's Modulus, Poisson's ratio, and average density. The Young's Modulus of the particles is the ratio of the stress, which has units of pressure, to strain, which is dimensionless. In one embodiment, the Young's Modulus may be about 10 to about 500 megapascals, and more preferably about 10 to about 100 megapascals and most preferably about 10 to about 30 megapascals. In a preferred embodiment herein, the Young's Modulus of the particles is preferably about 1 to about 500 megapascals, more preferably about 1 to about 100 megapascals and most preferably about 1 to about 30 megapascals.

The Poisson's ratio of the particles is another parameter that affects the ability of the particles to increase the fluid movement within a joint. Poisson's ratio is the ratio, when a sample is stretched, of the contraction or transverse strain (perpendicular to the applied load), to the extension or axial strain (in the direction of the applied load). As shown in Examples 1 and 2 described below, the preferable Poisson's ratio of the particles is about 0.1 to about 0.5. The Poisson's ratio is most preferably about 0.3.

The average density of the particles also contributes to the effectiveness of the particles in increasing fluid movement within the joint. The average density is preferably greater than the density of the fluid within the joint to reduce impingement in the joint interface. An average particle density greater that the density of the joint fluid also allows the particles to be positioned below the level of the joint fluid and thus “push” the fluid across the joint interface during joint motion. For example, the wringing action of the synovial capsule and upward stirring effect of the elliptically-shaped femoral neck facilitates this “pushing” action in a hip joint. The density of synovial fluid is typically about 1.015 g/ml. Accordingly, the average density of the particles is preferably greater than about 1.015 g/ml. The maximum density of the particles is preferably about 2.5 g/ml. The average density is most preferably about 1.2 g/ml.

The particles of the present invention are formed of at least one resorbable, biocompatible material(s) that is/are preferably commercially available and FDA-approved for use in the body of a mammal. As used herein, a resorbable material is defined as a material readily degraded in the body and subsequently disposed of by the body or absorbed into the body tissue. As used herein, a biocompatible material is one that is not toxic to the body and does not cause tissue inflammation. The particles of the present invention preferably resorb within the joint in about 3 to about 12 months, although the rate of resorbance will depend to some extent on the material chosen. The particles most preferably resorb in about 3 to about 6 months. As used herein, “mammal” encompasses humans and animals.

The resorbable, biocompatible particles of the present invention may be formed of natural or synthetic materials. The natural materials may include, among other materials, cat gut, cellulose, chitosan, carrageenan, starch, alginate, hyaluronic acid, and chitin. The synthetic materials preferably include polymers and copolymers, included cross-linked versions thereof. Non-limiting examples of resorbable, biocompatible polymers and elastomers suitable for making the particles of the present invention may include poly(alpha-hydroxy acid) polymers such as poly(glycolic acid) (PGA), copolymers of lactic acid and glycolic acid (PLGA), polyoxalates, polycaprolactone (PCL), copolymers of caprolactone and lactic acid (PCLA), poly(ether ester) multiblock copolymers based on polyethylene glycol and poly(butylene terephthalate), tyrosine-derived polycarbonates, poly(hydroxybutyrate), poly(alkylcarbonate), poly(orthoesters), polyesters, poly(hydroxyvaleric acid), poly(malic acid), poly(tartaric acid), poly(acrylamides), polyanhydrides, polyphosphazenes, and poly(dicarboxylic acid-polyol), such as poly(glycerol sebacate), poly(glycerol sebacate lactic acid), and copolymers and derivatives of the above materials. The polymers and copolymers may be random, alternating, block, or grafted polymers and copolymers and/or crosslinked polymers thereof. Suitable polymeric materials also include waxes such as glycerol mono- and distearate and the blends thereof. Such polymers may also be combined into blends, alloys or copolymerized or crosslinked with one another. The polymers in preferred embodiments herein can be elastomers and/or polymers having elastomeric properties and behavior.

Functional groups for specific properties (e.g., pH adjustment, or adjustment to physical properties or for crosslinking or surface modification) may be provided. Examples include, but are not limited to, alkyl, aryl, fluoro, chloro, bromo, iodo, hydroxyl, carbonyl, aldehyde, haloformyl, carbonate ester, carboxylate, carboxyl, ether, ester, hydroperoxy, peroxy, caroxamide, amine, ketimine, aldimine, imide, azide, diimide, cyanate, isocyanate, nitrate, nitrile, nitrosooxy, nitro, nitroso, pydridyl, sulfonyl, sulfo, sulfinyl, sulfino, sulfhydryl, thiocyanate, disulfide, phosphino, phosphono, phosphate groups, and combinations thereof. The preferred functional groups include carboxyl, alkyl ester, alkyl ether and hydroxyl groups. The more preferred functional groups include carboxyl and alkyl ester groups.

One class of preferred particle materials are copolymers of lactic acid and caprolactone. The most preferred material in this class being a copolymer of L-lactide and caprolactone such as poly(L-lactide-co-caprolactone) with an L-lactide to caprolactone monomer ratio of 70:30 or less. Suitable material is commercially available as PURASORB® PLC-7015 from Purac Biomaterials of Gorinchem, The Netherlands.

The inherent viscosity of the polymers and copolymers, measured in deciliters per gram, is a measure of the capability of the polymers and copolymers in solution to enhance the viscosity of the solution. The inherent viscosity is dependent upon the length of the polymer and copolymer chains and increases with increasing polymer or copolymer molecular weight. The inherent viscosity of the polymers or copolymers forming the particles is preferably about 0.15 deciliters per gram to about 3.0 deciliters per gram.

Another class of preferred particle materials is resorbable, biocompatible, polyester-based elastomers and/or polycarboxlic acid-polyol copolymers and elastomers as noted above, for example, PGS or PGSL. Suitable PGS materials are commercially available as Regenerez®, from Secant Medical, Inc. of Perkasie, PA, U.S.A., which may be formed, for example, in accordance with U.S. Pat. No. 9,359,472, relevant portions of which are incorporated by reference herein. Materials like PGS and PGSL can yield particles with enhanced properties as elastomeric materials due to a crosslinked structure.

One improvement is in the form of enhanced recovery in response to deformation, which allows the particle to retain the desired shape more effectively. A second improvement is in the form of enhanced retention of physical properties over the lifetime of the particle, in-vivo. Further, particles formed from PGS tend to erode from the outside in, rather than bulk erode, which means that the particles get smaller as they degrade, but retain their physical properties much longer than materials that degrade more homogenously throughout the bulk of the particle.

The particles may be formed of any shape including, but not limited to spherical, oval, elliptical, cuboidal, pyramidal, or cruciform. However, the particles are preferably spherical to minimize impingement in the joint interface.

The particles also may be formed of any known method for forming particles of the material and size described above. The particles are preferably formed via a melt-processing technique such as injection molding. Injection molding is a manufacturing process for producing articles from polymeric materials. The process includes first feeding the polymeric raw material into a container for heating. The resultant heated material is then mixed and added to a mold where it cools to form the particles of the present invention. Any other acceptable techniques for producing the particles of the present invention may be used including solvent-based processes such as double emulsion and solvent evaporation, freeze drying, spray drying, extrusion; cryoformation; or latex polymerization/separation. In the case of particles formed from crosslinked materials, such as PGS, the particles can be formed from uncrosslinked prepolymers and subsequently crosslinked to yield their final elastomeric form.

Additionally, as noted above, in a preferred embodiment, the bulk and/or surface of the particles can be further modified by various functional groups and/or by incorporating bio-lubricious compounds as noted above, including but not limited to lubricin or hyaluronic acid. The presence of bio-lubricious compounds on the surface of the particles in certain embodiments enhances frictional properties, resulting in improved movement within the joint and the mitigation of impingement. The presence of bio-lubricious compounds in the bulk of the particles, in the case of surface eroding materials such as PGS, can also provide replenishment of the bio-lubricious compounds as the particle degrades.

One method of incorporating the bio-lubricious compound into the particles is via grafting or other surface modification. Difunctional compounds, such as those used to crosslink bio-compatible hydrogels, can be used to connect bio-lubricious compounds to particles via reaction with functional groups present on the bio-lubricious compounds and on the polymers the particles are formed from. For example, both hyaluronic acid and the chondroitin sulfate moieties present on the terminal segments of lubricin contain hydroxyl and carboxylic acid groups that can be useful for grafting the molecules onto polymers useful for forming the particles of the invention. PGS, for example, being a polyester, also contains hydroxyl and carboxylic acid groups that can be exploited for the purpose of grafting reactions. Specific difunctional grafting agents include, but are not limited to glutaraldehyde, divinyl sulfone, adipic acid dihydrazide and butanediol diglycidyl ether.

The monomers used for forming the polymers may also include in a functionalized monomer or monomers preferred functional groups for receiving and reacting with lubricin, hyaluronic acid or the like for forming a copolymer having lubricin or hyaluronic acid bonded on various locations to a base polymer chain prior to particle formation and/or simply mixing such agents into the bulk of the material during or prior to formation of particles (such as through a latex or solvent reaction).

Another method of incorporating the bio-lubricious compound into the particles involves swelling the particles with a solution containing the bio-lubricious compound. Optionally, the solvent could subsequently be removed via evaporation to leave behind the bio-lubricious compound.

In another embodiment, a particle used herein may contain one or more of the resorbable, biocompatible materials described above and be coated with the same or a different resorbable, biocompatible material. For example, a particle of poly(L-lactide-co-caprolactone), PGS, PGSL or another resorbable biocompatible material can be formed with a coating, for example, an elastomeric PGS coating to achieve varying properties for different resorbance periods or different physical properties. A method of coating a particle with PGS is described for example in U.S. Patent Publication No. 2016/0251540 A1, incorporated herein in relevant part.

The present invention further includes a composition for increasing lubrication of a joint. The composition includes the particles described above and a carrier fluid. The carrier fluid may include, but is not limited to, aqueous solutions including physiologic electrolyte or ionic solutions such as saline solution or lactated ringer's solution, chondroitin sulfate, synovial fluid, viscosupplemental fluid such as hyaluronic acid commercially available as ORTHOVISC® produced by DePuy Ortho Biotech Products of Raritan, New Jersey, and combinations thereof. The composition may also include at least one therapeutic agent for treating osteoarthritis or other disease affecting the joints. The therapeutic agent may include hyaluronic acid, modified hyaluronic acid, anti-inflammatory medication such as steroids, non-steroidal anti-inflammatory agents, numbing agents such as lidocaine or the like.

The present invention further includes a method for lubricating a joint by introducing the particles described above into the joint. The particles may be introduced into the joint using any suitable device such as through a catheter, infusion pump, needle or a cannula. The particles are preferably introduced into the joint using a cannula with an inside diameter of about 2 millimeters to about 6 millimeters and more preferably about 4 millimeters to about 6 millimeters. The particles are preferably introduced into the joint by direct arthroscopic visualization, x-ray guided insertion, radiographically-guided insertion, sonographically-guided insertion or combinations thereof, although other known methods may also be used.

The number of particles introduced into the joint is dependent on the average size of the particles and the type of joint. The number of particles introduced into the joint is preferably an effective amount to increase fluid movement within the joint. In a typical hip joint, the volume of the synovial capsule is about 20 ml to about 200 ml. For an average particle size of about 3 millimeters, the number of particles introduced into the joint may include, but is not limited to, about 5 to about 1,000 particles. The number of particles is more preferably about 5 to about 100 particles.

The method may be used to lubricate any type of joint. However, the joint is preferably a synovial joint such as a hip, a knee, a shoulder, an ankle, an elbow, a wrist, a toe, a finger, and/or a spinal facet joint. The joint is more preferably a hip, ankle or knee joint. The method can also be used for lubricating a prosthetic implant or an arthritic or other diseased or injured joint. The method may further include introducing the particles in a carrier fluid including, but not limited to, aqueous solutions including physiologic electrolyte or ionic solutions such as saline solution or lactated ringer's solution, chondroitin sulfate, synovial fluid, viscosupplemental fluid, and combinations thereof and/or a therapeutic agent such as hyaluronic acid, modified hyaluronic acid, anti-inflammatory medication such as steroids, non-steroidal anti-inflammatory agents, numbing agents such as lidocaine or the like.

A method for treating a disease that causes irregularity of the joint surfaces or breakdown of the soft tissue in the joint such as osteoarthritis is also disclosed herein. The method includes introducing the particles described above into an arthritic joint, wherein the particles are capable of increasing fluid movement within the joint compared to synovial fluid, viscosupplemental fluid, or combinations thereof. The increase in fluid movement within the joint alleviates symptoms associated with osteoarthritis including pain and stiffness. Further, the use of the particles may forestall or eliminate the need for joint replacement surgery. The invention will now be illustrated in accordance with the following non-limiting examples.

### EXAMPLES

Example 1 illustrates the effectiveness of polymer particles in lubricating a joint compared with joint fluid alone. Example 2 evaluates polymers and copolymers to determine their suitability for forming the particles of the present invention.

### Example 1

A simulation was run on a three-dimensional model of a hip joint to evaluate the effectiveness of polymer particles in lubricating a joint compared with joint fluid alone. The model assumed the femur and the concave surface of the pelvis known as the acetabulum are rigid, the synovial capsule and particles are elastic, and the synovial fluid has similar properties to water. The capsule, fluid, and particle physical parameters are summarized in Table 1.

TABLE 1

| | | Young's | Poisson's |
|---|---|---|---|
| | Density | Modulus | Ratio |
| Material | (g/ml) | (MPa) | (dimensionless) |
| Capsule | 1.5 | 50 | 0.1 |
| Fluid | 1.0 | — | — |
| Particles | 1.5 | 2,360 | 0.3 |

Two simulations were run using Smooth Particle Hydrodynamics to evaluate the effectiveness of the particles in increasing the fluid movement and thus lubrication of a hip joint. The first simulation includes fluid particles only and assumes the fluid particles are located in the space between the synovial capsule and the neck of the femur and the space between the acetabulum and the femoral head. The second simulation includes fluid particles mixed with 3-mm diameter polymer particles. Prior to the simulation, the synovial capsule was shrunk by lowering its temperature to simulate pretension of the joint. The femur was then flexed forward 35 degrees, extended 60 degrees and finally returned to its original position. The kinetic energy and the number of fluid particles located between the femoral head and acetabulum were modeled at multiple times during the simulation. The results are shown in Table 2.

TABLE 2

| | Number of Fluid Particles | | Kinetic Energy of Fluid | |
|---|---|---|---|---|
| | Between Femoral Head and | | Particles Between Femoral | |
| | Acetabulum¹ | | Head and Acetabulum (N mm) | |
| | Without | With | Without | With |
| Time | Polymer | Polymer | Polymer | Polymer |
| (sec) | Particles | Particles | Particles | Particles |
| 0.000 | 1113 | 1113 | 0 | 0 |
| 0.040 | 2473 | 2394 | 0.128 | 0.224 |
| 0.0585 | 2527 | 2409 | 0.108 | 0.219 |
| 0.091 | 2573 | 2467 | 0.114 | 2.283 |
| 0.092 | 2560 | 2446 | 0.087 | 0.879 |

¹The total number of fluid particles in the capsule remained constant at 7,034 during the simulation. Fifty-one (51) polymer particles were added to the capsule for the second simulation.

The movement of the fluid particles was also monitored during the simulations. The fluid particles travelled longer distances within the joint when mixed with the polymer particles compared to the fluid particles alone.

Based on the simulations, addition of the polymer particles caused up to a 20-fold increase in the kinetic energy of the fluid particles within the hip joint. Further, the polymer particles increased the distance each fluid particle travelled while the hip was in motion. Accordingly, this Example demonstrates that the polymer particles can increase fluid movement in the joint, thereby, increasing lubrication of the joint.

### Example 2

Several FDA-approved, biocompatible, resorbable polymers and copolymers were tested to identify the preferred materials for forming the particles of the present invention. Various physical parameters of the polymers and copolymers were tested to evaluate suitability for forming the particles of the present invention. Table 3 identifies the materials.

TABLE 3

| Material | Tradename | Manufacturer | Comments |
|---|---|---|---|
| Poly | PURASORB ® PLC-8516 | Purac | 85:15 L- |
| (L-lactide-co-caprolactone) | | Biomaterials | lactide/caprolactone |
| | | | monomer ratio |
| Poly | PURASORB ® PLC-7015 | Purac | 70:30 L- |
| (L-lactide-co-caprolactone) | | Biomaterials | lactide/caprolactone |
| | | | monomer ratio |
| Poly | RESOMER ® LC-703-S | Boehringer- | 70:30 L- |
| (L-lactide-co-caprolactone) | | Ingelheim of | lactide/caprolactone |
| | | Ridgefield, | monomer ratio |
| | | Connecticut | |
| Poly | RESOMER ® RG-509-S | Boehringer- | 50:50 D, L- |
| (D,L-lactide-co-glycolide) | | Ingelheim | lactide/glycolide |
| | | | monomer ratio |
| Polydioxanone | RESOMER ® X-206-S | Boehringer- | Homopolymer |
| | | Ingelheim | |
| Poly | RESOMER ® R-207-S | Boehringer- | Homopolymer |
| (D,L-lactide) | | Ingelheim | |

The materials were first tested to determine whether the densities of the materials were greater than synovial fluid (i.e., 1.015 g/ml). Granules of each material were placed in a vial of a saline test solution with a density of 1.015 g/ml. All of the materials sank within the solution suggesting a density greater than that of synovial fluid. Accordingly, all the materials had a density greater than that of synovial fluid.

The materials were then tested in a synthetic synovial fluid test solution to simulate the properties of the samples within an osteoarthritic joint. Approximately 40 weight percent ORTHOVISC® was added to the saline test solution to simulate typical synovial fluid within an osteoarthritic joint based on a target viscosity of 1,400 centipoise at 25° C. Granules of each material were then added to a 1.0 ml vial of the synthetic synovial fluid test solution and allowed to equilibrate for three weeks. The samples were then analyzed via Differential Scanning calorimetry (DSC) from −40° C. to 90° C. to determine the T_{g}. The equilibrated T_{g} based on the DSC results and the melting temperature (Tₘ) and T_{g} of the dry materials are shown in Table 4.

TABLE 4

| | Dry Tₘ | Dry T_{g} | Equilibrated T_{g} |
|---|---|---|---|
| Sample | (° C.) | (° C.) | (° C.) |
| RESOMER ® | — | 50-60 | 47 |
| R-207-S | | | |
| RESOMER ® | 108-111 | 20 | * |
| LC-703-S | | | |
| RESOMER ® | — | 40-50 | 36 |
| RG-509-S | | | |
| RESOMER ® | 110 | −16 | * |
| X-206-S | | | |
| PURASORB ® | 108-111 | 20 | 9 |
| PLC-7015 | | | |
| PURASORB ® | 130 | 40 | 28 |
| PLC-8516 | | | |

* The RESOMER ® LC-703-S and RESOMER ® X-206-S yielded no clear T_{g} over the DSC temperature range.

The samples with an equilibrated T_{g} lower than body temperature of 37° C. based on DSC results were then qualitatively tested for stiffness. The previously equilibrated samples with T_{g} lower than 37° C., RESOMER® RG-509-S, PURASORB® PLC-7015 and PURSORB® PLC-8516, were heated to 37° C. for 24 hours. The samples were then examined with a spatula to qualitatively evaluate the stiffness of the materials. The PURASORB® PLC-7015 was elastic after heating while the other two materials were inflexible and likely unsuitable due to potential impingement in the joint after implantation. The physical properties of the PURASORB® PLC-7015 are reproduced in Table 5.

TABLE 5

| | Density | Young's Modulus | |
|---|---|---|---|
| Sample | (g/ml) | (MPa) | Poisson's Ratio |
| PURASORB ® | 1.22 | 30 | 0.3 |
| PLC-7015 | | | |

As illustrated in these Examples, the present invention fulfills a need in the art for an innovative method to improve joint lubrication and thus address the degradation and reduction of synovial fluid associated with aging, osteoarthritis, injuries and other ailments.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. Articles for increasing lubrication of a joint comprising:
resorbable, biocompatible particles capable of increasing fluid movement within the joint compared to synovial fluid, viscosupplemental fluid, or combinations thereof, wherein the particles comprise at least one polymer selected from polylactide-co-caorolactone), poly(glycerol sebacate), and poly(glycerol sebacate lactic acid) and have an average particle size of about 0.5 millimeters to about 5 millimeters.

2.-3. (canceled)

4. The articles of claim 1, wherein the particles have a Young's Modulus of about 1 megapascal to about 500 megapascals, a Poison's ratio of about 0.1 to about 0.5 and/or an average density that is greater than an average density of a fluid within the joint.

5.-9. (canceled)

10. The articles of claim 4, wherein the average density of the particles is about 1 g/ml to about 2.5 g/ml.

11. (canceled)

12. The articles of claim 1 having a glass transition temperature within a joint of less than about 37° C.

13. The articles of claim 1, wherein the particles resorb in vivo in about 3 to about 18 months.

14.-20. (canceled)

21. The articles of claim 1, wherein the particles comprise poly(glycerol sebacate).

22. The articles of claim 1, wherein the particles are elastomeric and/or the at least one polymer in the particles incorporates at least one bio-lubricious compound selected from lubricin and hyaluronic acid.

23.-26. (canceled)

27. The articles of claim 1, wherein the particles are generally spherical.

28. A composition for increasing lubrication of a joint comprising:
resorbable, biocompatible particles formed of at least one resorbable biocompatible polymer, wherein the particles are capable of increasing fluid movement within the joint compared to synovial fluid, viscosupplemental fluid, or combinations thereof, wherein the particles comprise at least one polymer selected from poly(L-lactide-co-caprolactone), poly(glycerol sebacate), and poly(glycerol sebacate lactic acid) and wherein the particles have an average particle size of about 0.5 millimeters to about 5 millimeters; and
a carrier fluid.

29. The composition of claim 28, wherein the carrier fluid comprises saline solution, lactated ringer's solution, chondroitin sulfate, synovial fluid, viscosupplemental fluid, and combinations thereof.

30. The composition of claim 28, wherein the composition further comprises at least one therapeutic agent selected from hyaluronic acid, modified hyaluronic acid, anti-inflammatory medications, non-steroidal anti-inflammatory agents, a numbing agents, and combinations thereof.

31.-33. (canceled)

34. The composition of claim 28, wherein the at least one polymer comprises poly(glycerol sebacate).

35.-40. (canceled)

41. A method of lubricating a joint comprising introducing particles into a joint, wherein the particles are capable of increasing fluid movement within the joint compared to synovial fluid, viscosupplemental fluid, or combinations thereof and are formed of a resorbable, biocompatible material comprising a polymer selected from the group consisting of poly(L-lactide-co-caprolactone, poly-(glycerol sebacate), and poly(glycerol sebacate lactic acid).

42. The method according to claim 41, wherein the resorbable biocompatible material comprises an elastomeric poly(glycerol sebacate).

43. The method according to claim 41, wherein the resorbable biocompatible material has a glass transition temperature within the joint of less than about 37° C.

44. The method according to claim 41, further comprising introducing the particles into the joint through a cannula.

45. The method according to claim 44, wherein an inside diameter of the cannula is about 2 millimeters to about 6 millimeters.

46. (canceled)

47. The method according to claim 41, further comprising introducing the particles into the joint by arthroscopic visualization, x-ray-guided insertion, radiographically-guided insertion, sonographically-guided insertion or combinations thereof.

48. The method according to claim 41, wherein the joint is a synovial joint, selected from one or more of a hip, a knee, a shoulder, an ankle, an elbow, a wrist, a toe, a finer, and a spinal face joint.

49. (canceled)

50. The method according to claim 48, wherein the joint is a prosthetic implant.

51. The method according to claim 48, wherein the joint is an arthritic joint.

52. The method according to claim 41, wherein an average particle size is about 0.5 millimeters to about 5 millimeters.

53.-54. (canceled)

55. The method according to claim 41, further comprising introducing the particles into the joint with a therapeutic agent and/or a carrier fluid comprising saline solution, lactated ringer's solution, chondroitin sulfate, synovial fluid, viscosupplentental fluid, and combinations thereof.

56. The A method of claim 41, further comprising incorporating at least one biolubricious compound into the polymer.

57. The method according to claim 56, wherein the at least one biolubricious compound is incorporated into the polymer by: (a) reacting an at least one reactive functional group of the polymer by a grafting and/or surface modification reaction using a difunctional compound to crosslink the at the least one functional group on the polymer with a functional group on the at least one biolubricious compound at the surface of the particle; or (b) by forming the polymer using at least one functionalized monomer capable of reacting with the at least one biolubricious compound so as to attach the at least one biolubricious compound to at least one location along a chain of the polymer before forming the particles.

58. (canceled)

59. The method according to claim 57, wherein when the biolubricious compound is incorporated by forming the polymer according to (b), the at least one biolubricious compound is combined with the resorbable biocompatible material in a solvent-based reaction or latex polymerization reaction.

60. The method according to claim 56, wherein the at least one biolubricious compound is combined with the resorbable biocompatible material prior to formation of the particles through at least one of mixing and/or blending.

61. The method according to claim 56, wherein the at least one biolubricious compound is combined with the resorbable biocompatible material by swelling the particles with a solution comprising the biolubricious compound.

62. The method according to claim 41, wherein the particles are formed by at least one of a melt-processing process; a thermally cured condensation reaction process; a polymerization process initiated thermally or initiated by irradiation with ultraviolet, e-beam, gamma or other radiation; a solvent-based process; cryoformation; or latex polymerization.

63.-76. (canceled)

77. The method according to claim 41, wherein joint is a diseased joint and the method further comprises treating a disease that causes irregularity of the joint surfaces or breakdown of the soft tissue in the joint, and wherein the particles are introduced the diseased joint.

78. (canceled)

79. The method according to claim 77, wherein the disease of the diseased joint is osteoarthritis.
